(19) United States
(12) Patent Application Publication
(10) Pub. No.: US 2022/0313982 A1
(43) Pub. Date: Oct. 6, 2022

(54) WEARABLE DEVICE TO AMELIORATE PAIN

(71) Applicant: Stephan Ryan Martinez, Seattle, WA (US)

(72) Inventor: Stephan Ryan Martinez, Seattle, WA (US)

(21) Appl. No.: 17/712,382

(22) Filed: Apr. 4, 2022

Related U.S. Application Data
(60) Provisional application No. 63/170,657, filed on Apr. 5, 2021.

(51) Int. Cl. A61N 1/04; A61N 1/36; A61N 1/02

(52) U.S. Cl. CPC A61N 1/0484; A61N 1/0476; A61N 1/36021; A61N 1/025; A61N 1/36034

(57) ABSTRACT

The present invention describes a wearable device that is designed to ameliorate pain. The wearable device has been designed and developed to provide low-level random pattern electrical stimulation to skin in order to mask the associated pain. The device is intended to allow a better semblance of normalcy with less pain. The wearable device may be a sleeve with a plurality of electrodes with electrically conductive media that provides random stimulation to the skin. The wearable device may include wearable fabric lined with a conductive medium, a power supply medium such as a battery set, a processor to handle input/output (I/O) from/to a mobile application or controller, a transmitter, a receiver, and motion sensors. With the wearable device disclosed herein, the goal is to have this work on many parts of the user's body from extremities to the trunk.

### CROSS-REFERENCE TO RELATED PATENT DOCUMENTS

This patent application claims the benefit of priority of U.S. Provisional Application No. 63/170,657, entitled “WEARABLE DEVICE TO AMELIORATE PAIN,” filed Apr. 5, 2021, which are hereby incorporated herein by reference in its entirety.

### TECHNICAL FIELD OF THE INVENTION

The present invention relates in general to the field of a medical device, and, more particularly, to a wearable device that is designed to ameliorate pain. The wearable device has been designed and developed to provide low-level random pattern electrical stimulation to skin in order to mask the pain associated with a body part of a user. The disclosed wearable device, without limiting the scope of the present invention, may be construed as a sleeve with a plurality of electrodes with electrically conductive media, a power supply and controller, and control software, that provides random stimulation to the skin.

### BACKGROUND OF THE INVENTION

In general, medical professionals rely on numerous treatment options for managing patients' pain as treatment for chronic conditions and persistent pain. The most common treatment option is medication. Pain reducing medications, in combination with muscle relaxants, tranquilizers, and steroids, are commonly prescribed for patients as part of a pain reduction regimen. However, prolonged use of medication is known to have adverse medical side effects, which often require patients to stop taking medication or to continually increase the amount of medication used to obtain the same level of pain reduction. Alternatively, or in combination with medication, electronic stimulation devices have been found to encourage improved physiological conditions that promote improved healing times and effectively reduce pain levels. Therapies, including heat, compression, and vibration, have also been found to reduce pain and promote wound healing.

The stimulation device can provide a touch-like sensation across the dermis significantly decreasing the discomfort and pain associated with the chronic ailments. Users who experience this type of pain, especially those suffering from adhesive capsulitis, tendonitis, muscle strains, tendon strains, ligament strains, or the like, are affected not only by pain but suffer the pernicious by products as a result of such pain. Users with chronic pain, especially those with ailments described above experience difficulty sleeping and debilitating pain when walking due to a lack of stimulation near the time of waking. These issues lead to negative consequences for mental and physical health. This stimulation device was invented in order to offer a sufferer of chronic pain a peaceful night of sleep with little to no residual pain when walking as well as daily use for therapeutic purposes. There are also other uses past these in the areas of virtual reality enhancement, video game/experience enhancement, audio and visual media enhancement, remote sensing, remote therapy, and sexual stimulation. Traditional Transcutaneous Nerve Stimulation (“TENS”) and Electronic Muscle Stimulation (“EMS”) units have been employed to manage pain and discomfort of this kind with varying degrees of electrical pulses. The disadvantage of this traditional method includes (but is not limited to) discomfort at all electrical pulse levels, limited functionality, limited session time, limited pattern choices, complicated and time-consuming wiring apparatuses, bulky devices, and non-“smart” functionality. In addition, all these existing avenues focus on the sub-dermal transmission which this device is not intending to accomplish.

Currently, there are various products, such as Icy Hot Smart Relief™, Copper Fit®, Phiten©, or the like, in the market that can be used for relieving the pain, but each come with its own advantages and disadvantages. For example, Icy Hot Smart Relief™ is simple to use and simple to apply but it requires additional pads over time, and it has low battery life. Copper Fit® is a cheap product and is simple to use and apply but it is highly inefficient and thus is not reliable. Similarly, other commercial products are not reliable and are highly inefficient.

In addition to the above prior art products, there are few patent references that disclose similar pain-relieving devices. For example, PCT patent publication number 2020047175 discloses a device including a casing having an application area for transmitting vibration and thermal effects, at least one vibratory source, at least one power source, at least one switch to actuate the vibratory source, electrical communication between the vibratory source, the power source, and the switch, and a thermal element for modulating the temperature of a contacted area of a surface. US patent publication number 2019110950 discloses methods of operating a haptic actuator to apply physical and/or thermal sensations to a user to provide subjective symptom relief to a user are described.

All the above-mentioned prior arts disclose different methods and devices for use in relieving the one or more types of pain. The related products may be easy to use or apply but are not efficient and effective. They either don't work at all or even if some efficacy, there is a Faustian bargain especially with the analgesic drugs and none of them are curative or restorative, they only mask pain, and hence, may not be truly helpful. In light of the foregoing, the present invention focuses on a wearable device that efficiently works to ameliorate the chronic pain.

### BRIEF SUMMARY OF THE INVENTION

To minimize the limitations of the prior art, and to minimize other limitations that will be apparent upon reading and understanding the present specification, the present invention discloses a wearable device that is designed to ameliorate pain. The wearable device has been designed and developed to provide low-level random pattern electrical stimulation to skin in order to ameliorate the pain associated with chronic pain conditions such as frozen, painful, or uncomfortable shoulder. The device is intended to allow a better semblance of normalcy with less pain. Without limiting the scope of the present invention, the wearable device may be considered as a sleeve with a plurality of electrodes with electrically conductive media, a power supply and controller, and control software, that provides random stimulation to the skin. The wearable device may include wearable fabric lined with a conductive medium, a power supply medium such as a battery set, a processor to handle input/output (I/O) from/to a mobile application or controller, a transmitter, a receiver, and motion sensors. With the wearable device disclosed herein, the goal is to have this work on many parts of the user's body from extremities to the trunk. A non-comprehensive list may include, but is not limited to, wrist, elbow, shoulder, ankle, calf, knee, thigh, lower back, neck, or the like.

One object of the present invention discloses a wearable device that is designed and configured for simulating physical sensation. In an embodiment, the wearable device comprises a fabric and closure system for securing to a user. The wearable device further comprises a configuration featuring wires along all sides that bridge electrical connections under the fabric. The wires are inside material construction layers with nothing visible to a naked eye. The wearable device further comprises a movement sensor system. The sensor system comprises a plurality of movement sensors to track movements of the user allowing the device to properly stimulate. The wearable device further comprises a battery set including one or more rechargeable batteries or others that are configured to power the device. The wearable device further comprises a processor that is configured to receive directive(s) from a transmitter. The processor is further configured to interpret the directive(s) and organize stimulation accordingly. The processor is further configured to send the stimulation to chosen wires. The processor is further configured to terminate stimulation when the circuit is broken. The processor is further configured to gather and send a battery charge report and the set of sleeping patterns from an internal recording or via third-party integration. The wearable device further comprises a transmitter and receiver for remotely communicating with a mobile device application or remote device and processor relaying a user selection of a plurality of operating modes. The operating directive corresponds to the selected mode and includes at least one of a plurality of pulse frequencies, a plurality of pulse-widths, a plurality of durations, and a plurality of signals. The signals may be randomly distributed by changing the connection between various electrodes.

In an embodiment, the processor, the transmitter, the receiver, the movement sensor system, and the battery are coupled to the outside of the fabric. In an embodiment, the processor is able to vary the energy, pulse width, frequency, duration, and current of the stimulation. In an embodiment, the movement pattern reports of the user defines a movement data set. In an embodiment, the processor sensor is configured to obtain multiple movement data sets over multiple time instances and communicate said movement set(s) to the transmitter. In an embodiment, the movement sensing system on board or via third-party integration is configured to obtain movement data from a single session. The data will be compared and cumulated in the user movement data set communicated from the transmitter to a mobile device application or remote. In an embodiment, the movement sensing data is obtained before, during, and after session for comparative analysis. In an embodiment, the processor is capable of delivering a specific dermal stimulation mode through a wireless transmission from a signal originating from a mobile device application or remote via the transmitter/receiver system. The processor is capable of implementing a mode selected from a plurality of modes consisting of a basic on and off setting, a mode consisting of a timed and timer setting, a mode consisting of using the movement sensing system, a mode communicating with a third-party wearable device, and others. The processor is configured to communicate with the third-party wearable device. The wearable device contains the ability to track movement of the user. In an embodiment, the processor contains the ability to provide feedback personalized to the user about health, sleep, and movement presented through a mobile device application or remote. The transmitter and receiver system communicates directly with a mobile device application or remote via Bluetooth capabilities.

Another object of the present invention discloses a method for monitoring the sleep and movement patterns of the user. The movement monitoring method comprises various features or processes that are executed by one or more components. For example, a movement sensor system with an ability to sense movements as they apply to sleep and rest, a processor configured to define data gathered and properly respond to movement in relation to a sleep cycle of the user with a corresponding change in stimulation, and a transmitter and receiver system in communication with the processor to properly communicate the data to a mobile device application or remote. The movement sensor system compiles, analyses, and shares data. The processor cumulates the data from the movement sensor system and assembles a user-specific database. The receiver acquires the signal from the mobile device application or remote and is transferred to the processor. The processor sends signals and data from the device to the transmitter which in turn is presented on a mobile device application or remote.

Another object of the present invention discloses a wiring system of a wearable device simulating physical sensation. The system comprises an arrangement of wires inside and around all sides of the fabric. The system further comprises a battery rechargeable or other that supplies energy. The system further comprises a processor that is configured to send stimulation across chosen wires. The processor further configured to receive directive(s). The processor is further configured to respond to said directive(s) by delivering corresponding stimulation. The processor further configured to be powered by the battery source. The system further comprises a conductive pathway or other mechanism such as haptic, sonic or other inside and around all sides of the fabric meant to physically contact the user. The conductive pathway or other mechanism such as haptic, sonic or other that send and receive impulses of varying strength level and frequency across the dermis of the user.

Various embodiments of the present invention present multiple advantages to other products and services presented to solve the issue of the chronic pain. The simplicity of the present embodiment imparts little inconvenience or cost to the user. The only additional product required along with the device is a remote or smart device application which allows the device to execute therapeutic experiences customized for and by the user. Such experiences prove to be easy, comfortable, convenient, and discreet. Compared to medical solutions, the present embodiments allow the experience to be informal and unobtrusive. Other traditional TENS and EMS devices send electricity through the epidermis and dermis deeper into the body, the present embodiments of the invention, contrarily, run stimulation across the dermis, allowing for minimal to no penetration of the epidermis and dermis. The experience of the present embodiments incurs less expense on the user and/or insurance provider. The present embodiments allow for such issues to be significantly quelled.

Various other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

The novel features which are believed to be characteristic of the present invention, as to its structure, organization, use, and method of operation, together with further objectives and advantages thereof, will be better understood from the following drawings in which a presently preferred embodiment of the invention will now be illustrated by way of various examples. It is expressly understood, however, that the drawings are for the purpose of illustration and description only and are not intended as a definition of the limits of the invention. Embodiments of this invention will now be described by way of example in association with the accompanying drawings in which:

FIG. 1 is a diagram that illustrates a wearable device simulating physical sensation including electrical hardware, accompanying technology, fabric, and closure system necessary, according to an exemplary embodiment of the present invention.

FIG. 2 is a diagram that illustrates an internal stimulation wiring pathway structure of the wearable device simulating the physical sensation, according to an exemplary embodiment of the present invention.

FIGS. 3A-3D are diagrams that illustrate an exemplary wearable device developed to treat a painful or uncomfortable body part of a user, according to an exemplary embodiment of the present invention.

FIG. 4A is a diagram that illustrates a wearable device to ameliorate pain, according to another exemplary embodiment of the present invention.

FIG. 4B is a diagram that illustrates an exploded version of the wearable device, according to another exemplary embodiment of the present invention.

FIG. 4C is a diagram that illustrates a use case scenario of using the wearable device to ameliorate pain, according to another exemplary embodiment of the present invention.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

Before describing the present invention in detail, it should be observed that the present invention utilizes a combination of components, which constitutes a wearable device that is designed to ameliorate pain. The wearable device has been designed and developed to provide low-level random pattern electrical stimulation to the skin in order to mask the pain. The wearable device may be a sleeve with a plurality of electrodes with electrically conductive media, a power supply, and controller, and control software, that provides random stimulation to the skin. Accordingly, the components have been represented, showing only specific details that are pertinent for an understanding of the present invention so as not to obscure the disclosure with details that may be readily apparent to those with ordinary skill in the art having the benefit of the description herein. As required, the detailed embodiments of the present invention are disclosed herein. However, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

The wearable device to ameliorate pain will now be described with reference to the accompanying drawings, which should be regarded as merely illustrative without restricting the scope and ambit of the present invention.

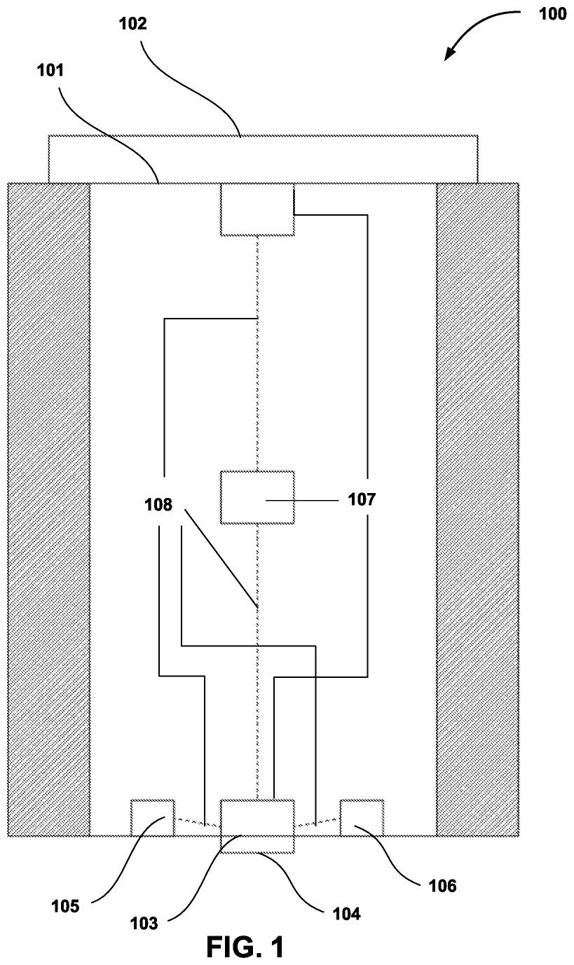

FIG. 1 is a diagram that illustrates a wearable device simulating physical sensation 100 including electrical hardware, accompanying technology, fabric, and closure system necessary, according to an exemplary embodiment of the present invention. In FIG. 1, an exemplary embodiment of the wearable device simulating physical sensation 100 for therapeutic and other sensation uses has been shown without limiting the scope of the present invention. In an embodiment, the wearable device 100 includes a fabric 101, a closure system 102, a battery set 103, a charging port 104, a processor 105, a transmitter/receiver system 106, movement sensors 107, and an electrical system 108. The primary purpose of this device 100 is to stimulate nerves and other structures in the epidermal and dermal layers, which will not go deeper into muscular structures. The processor 105 may be configured to receive directives from the transmitter 106, interpret the directives and organize the stimulation accordingly, send the stimulation to chosen wires, terminate the stimulation when the circuit is broken, and/or gather and send a battery charge report associated with the battery and a set of sleeping patterns from internal recording or via third-party integration. The transmitter/receiver system 106 is configured for remotely communicating with a mobile device application relaying a user selection of a plurality of operating modes. The operating directive corresponds to the selected mode and includes at least one of a plurality of pulse frequencies, a plurality of pulse-widths, a plurality of durations, or a plurality of signals.

The fabric 101 may be shaped to fit around an extremity of a user or a body section such as lower-back or upper-back. The fabric 101 is made of a flexible elastomeric material which, when applied, draws snugly and conforms about the contours of such extremity or body section. The fabric 101 may have the ability to draw snugly around most extremities or locations on account of the closure system 102 on both sides of the fabric 101. The closure system 102 is a fastener, for example, a version of the hook-and-loop fastener, more commonly known as Velcro® but should not be construed as limiting to the scope of the present invention. Other fasteners may include a dual lock fastener or a tension strap system or any other fastener that is effective and efficient with the disclosed application herein. The fit of the fabric 101, and hence the functionality of the wearable device 100 may be dependent on the closure system 102. There are other closure devices that may be implemented for varying locations and use case scenarios. As shown in FIG. 1, upon the exemplary embodiment of the wearable device simulating physical sensation 100 is the battery set 103 including one or more rechargeable batteries (hereinafter, may be referred to as the battery 103). The battery 103 may be located on the outside of the fabric 101 to maximize comfort while in use. The battery 103 may have the ability to be recharged over the lifespan of the wearable device 100. The battery 103 may be connected to the charging port 104 that will allow the battery 103 to be recharged multiple times over the lifespan of the wearable device 100.

Although not shown, the charging cable may be provided that connects to the battery 103 when not in use and is sold along with the device 100. The charging cable may be used to power the battery 103 from an external power supply, which in turn charges the battery 103 when not in use. The battery 103 may be configured to power the wearable device 100 in general including all of its communicating, sensing, processing, and stimulation functionalities such as one or more processors to handle input/output (I/O) from/to a mobile application or controller, one or more transmitters, one or more receivers, and one or more motion sensors. In some alternative embodiments, the battery 103 may also be a disposable version that is replaced when diminished.

As shown in FIG. 1, the exemplary embodiment of the wearable device simulating physical sensation 100 includes the processor 105. The processor 105 may be located on the outside of the fabric 101 to maximize comfort while in use. The processor 105 may be powered by the battery 103. The processor 105 proves integral in the functionality of the wearable device 100. The ability of the processor 105 may include, but is not limited to, sending signals, recognizing and interpreting movement data, compiling and managing movement data, and communicating with the transmitter/receiver system. For example, the processor 105 may be configured to receive directive(s) from a transmitter of the transmitter/receiver system 106. The processor 105 may be further configured to interpret the directive(s) and organize stimulation accordingly. The processor 105 may be further configured to send the stimulation to chosen wires. The processor 105 may be further configured to terminate stimulation when the circuit is broken. The processor 105 additionally monitors the health of the battery 103 in order to properly notify the user when there is a need for charging or replacement, depending on the version of said battery 103. The processor 105 may be further configured to gather and send a battery charge report and the set of sleeping patterns from an internal recording or via third-party integration. The processor 105 may be further configured to vary the energy, pulse width, frequency, duration, and current of the stimulation. The processor 105 may be further configured to deliver a specific dermal stimulation mode through a wireless transmission from a signal originating from a mobile device application or remote via the transmitter/receiver system 106. The processor 105 may be further configured to implement a mode selected from a plurality of modes consisting of a basic on and off setting, a mode consisting of a timed and timer setting, a mode consisting of using the movement sensing system, a mode communicating with a third-party wearable device, and others. The processor 105 may be further configured to communicate with the third-party wearable device. The wearable device contains the ability to track movement of the user. In an embodiment, the processor 105 contains the ability to provide feedback personalized to the user about health, sleep, and movement presented through a mobile device application or remote.

As shown in FIG. 1, the exemplary embodiment of the wearable device simulating physical sensation 100 includes the transmitter/receiver system 106. The transmitter/receiver system 106 may be located on the outside of the fabric 101 to maximize comfort while in use and is powered by the battery 103. The transmitter/receiver system 106 communicates with a remote or smart product running an application. Such system 106 and application or remote may transfer data in order to commence, perform, and cease stimulation therapy. The data transmitted and received includes but is not limited to: selections from a plurality of operating modes chosen by the user, a plurality of frequencies chosen by the user, a plurality of pulse-widths chosen by the user, a plurality of durations chosen by the user, a plurality of patterns (either chosen by the user or initiated by the user), raw and compiled movement sensor data, battery health and charge/replacement alerts, initiation of electrical signals, and cessation of electrical signals.

As shown in FIG. 1, the exemplary embodiment of the wearable device simulating physical sensation 100 further includes multiple movement sensors 107. The movement sensors 107 may be located on the outside of the fabric 101 to maximize comfort while in use and is powered by the battery 103. The amount of movement sensors 107 depend on the various embodiments the device takes. Regardless of the specific amount of movement sensors 107, the purpose of these sensors is to detect the movement of the user while the wearable device 100 is both attached to the user and functioning in a certain mode, both conditions relate to the exemplary embodiment of the wearable device 100. Although the main purpose of the sensors 107 and accompanying mode carried out via smart product application or remote is to monitor sleep cycle patterns and correspond said cycle with proper stimulation in order to limit pain experienced by the user, it can generally be used for occasions when the user anticipates a period of inactivity for corresponding pain area. In general, the multiple movement sensors 107 detect user body movement data. Such detected data is sent to the processor 105 which in turn translates the data into decisions based on the hardware, processor, and software of the mobile device application or remote. Such data is both simultaneously translated into a change of power and/or frequency and/or pattern of stimulation. The data is sent to the transmitter 106, relaying the change of power to the smart product application or remote. The movement sensor system has been created in order to reduce the effects that the users may experience when they are immobile for long periods of time. The system is designed to aid the user in sleeping or relaxation, in this exemplary embodiment. The stimulation would allow the user to enter or re-enter a state of relaxation and/or sleep. Once the movement sensor system would detect a lack of user movement, the stimulation would gradually decrease the power and/or change frequency and/or pattern. A quiet period would ensue when the stimulation would cease caused by the processor 105, however the movement sensor system would remain active. Once the movement sensor system would detect a change in immobility, usually characterized by the later stages of the sleep cycle, the system would utilize the processor 105 to phase the stimulation in, gradually increasing power. This could also be linked to third-party applications that log, categorize, and predict wakefulness and sleep patterns. This would, in essence, prime the affected area for movement after a prolonged immobile interval. In the exemplary embodiment of the device 100, the multiple movement sensors 107 are instrumental in the movement sensing system.

As shown in FIG. 1, inside the exemplary embodiment of the wearable device simulating physical sensation 100 is the electrical system 108. The electrical system 108 may be located on the outside the fabric 101. The electrical system 108 transmit signals from the battery 103 to the different technology sources located on the wearable device 100. The electrical system 108 specifically provides power to the processor 105, the transmitter/receiver system 106, the movement system sensors 107, and other areas depending on the embodiment of the present invention. The electrical system 108 also allows each technology source to communicate to other sources. The electrical system 108 allows the processor 105 to consolidate and respond to data captured by the movement sensor system 107. It also allows the battery 103 to report on its charge and health to the processor 105, and it allows the transmitter/receiver to have open communication with the processor 105 concerning certain types of stimulation instructed, decisions based on the data captured by the movement sensor system 107 and the health and charge of the battery 103. Overall, the electrical system 108 allows the device 100 to work as a smart device, allowing communication between the device 100 and another smart device or remote as well as allowing for user customization and selection of modes.

In some embodiments, the signals may be randomly distributed along the body part by changing the connection of various electrodes. This mechanism may be referred to as the random nerve stimulation that may be critical and useful to pain alleviation. The random nerve simulation may be achieved by preforming the rotation of various electrode pairs and firing them based on their connection.

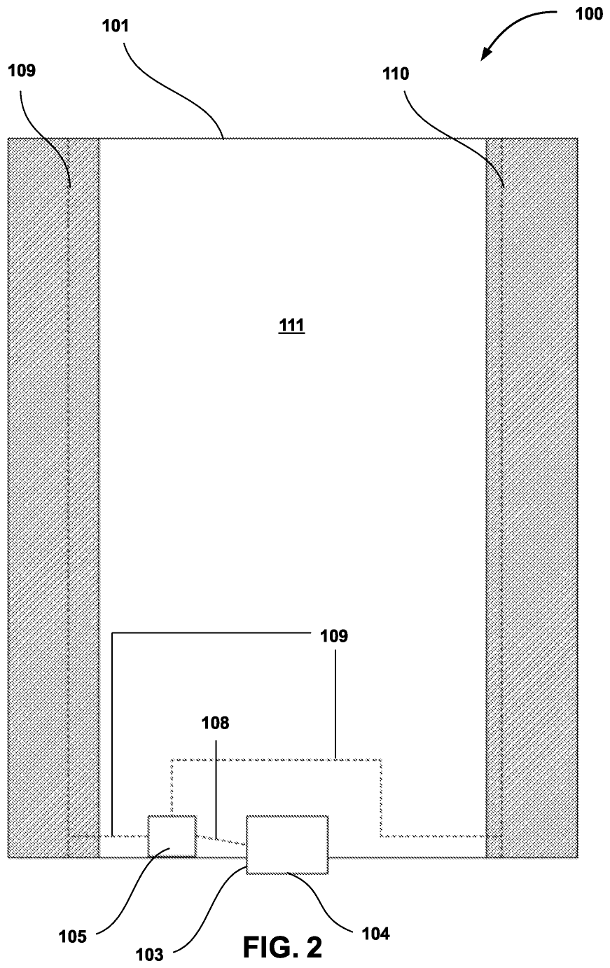

FIG. 2 is a diagram that illustrates an internal stimulation wiring pathway structure of the wearable device simulating the physical sensation 100, according to an exemplary embodiment of the present invention. As shown in FIG. 2, the wearable device simulating physical sensation 100 may include a conductive pathway system 109. The conductive pathway system 109 may be located on the outside and underside the fabric 101. The conductive pathway system 109 may allow signals to be sent from the processor 105 to one side of the device 100. The conductive pathway system 109 then splits off many into a plurality of ends, all penetrating through the fabric sending signals onto a conductive grid system 111 on the underside of the fabric 101. As further shown in FIG. 2, the wearable device simulating physical sensation 100 further includes a ground system 110. The ground system 110 physically opposes the physical location of where the conductive pathway connects with the conductive grid system 111. The ground system 110 takes in the signal released on the grid system 111 from the conductive pathway system 109. Once the signal reaches the ground system 110, the signal will vacate the grid system 111 and the journey of the signal will be complete. As shown in FIG. 2, on the underside of the exemplary embodiment of a wearable device simulating physical sensation 100 is a conductive grid system 111. When the device 100 is properly in use, the conductive grid system 111 will be in direct contact with the dermis of the user. When a signal reaches the conductive grid system 111 via the conductive pathway system 109, the signal will travel across the system 111 eventually reaching the ground system 110. Note, the grid pattern 111 described and illustrated in the detailed description and accompanying illustration(s) is in reference to the exemplary embodiment. The device is not limited to type, shape, or appearance of the conductive grid system 111.

The stimulation system and method of the present invention therefore provide a compact stimulation device that can operate in a plurality of modes to adapt to an individual user's physiology, target pain area, and desired treatment mode. In particular treatment modes, the stimulation device can monitor a treatment session and provide appropriate feedback to a user based upon delivered stimulation and detected muscle feedback and response. The stimulation device can therefore be used safely and effectively by non-medical personnel for training and therapeutic applications.

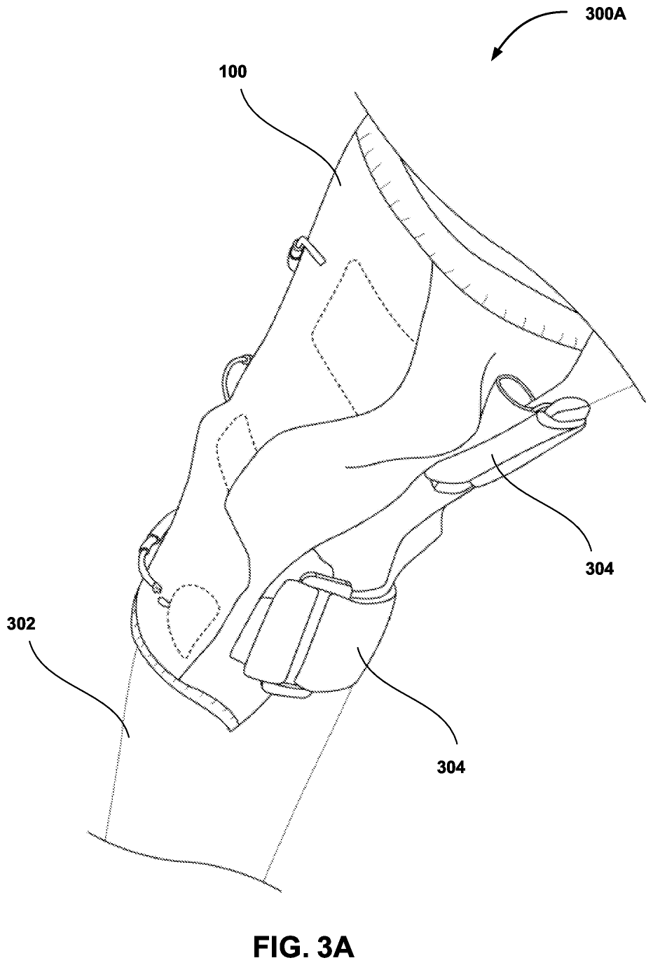

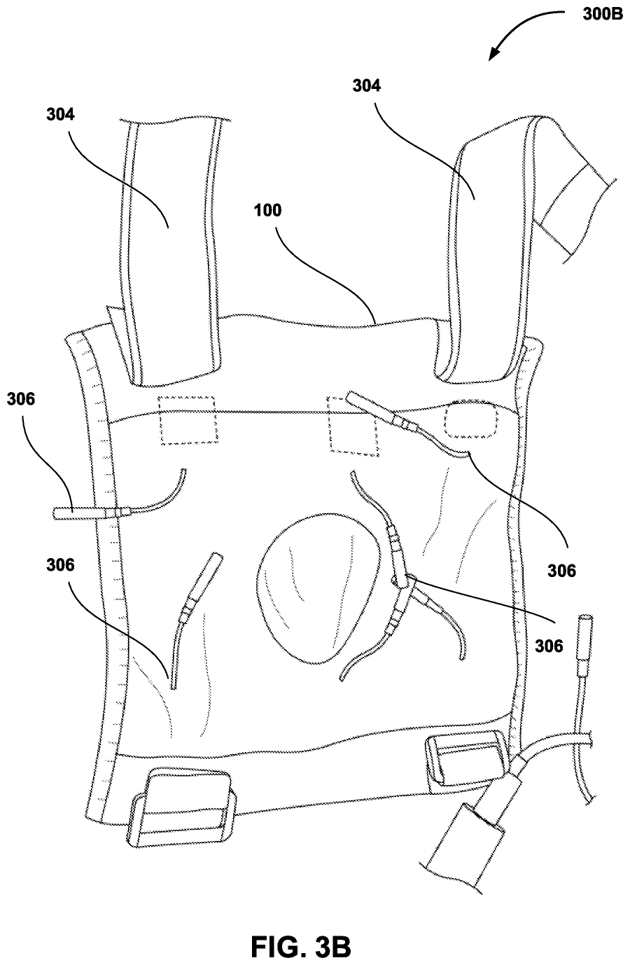

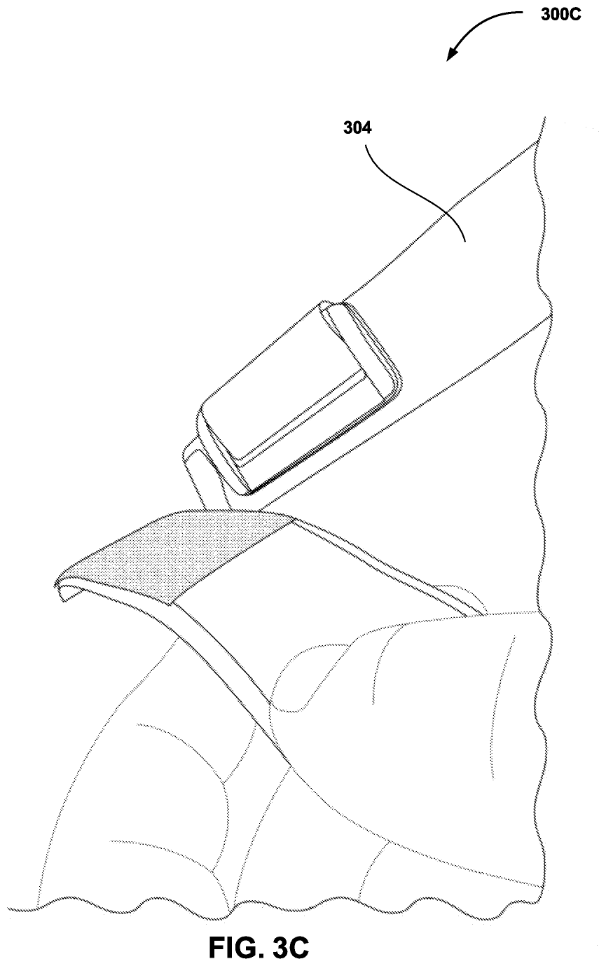

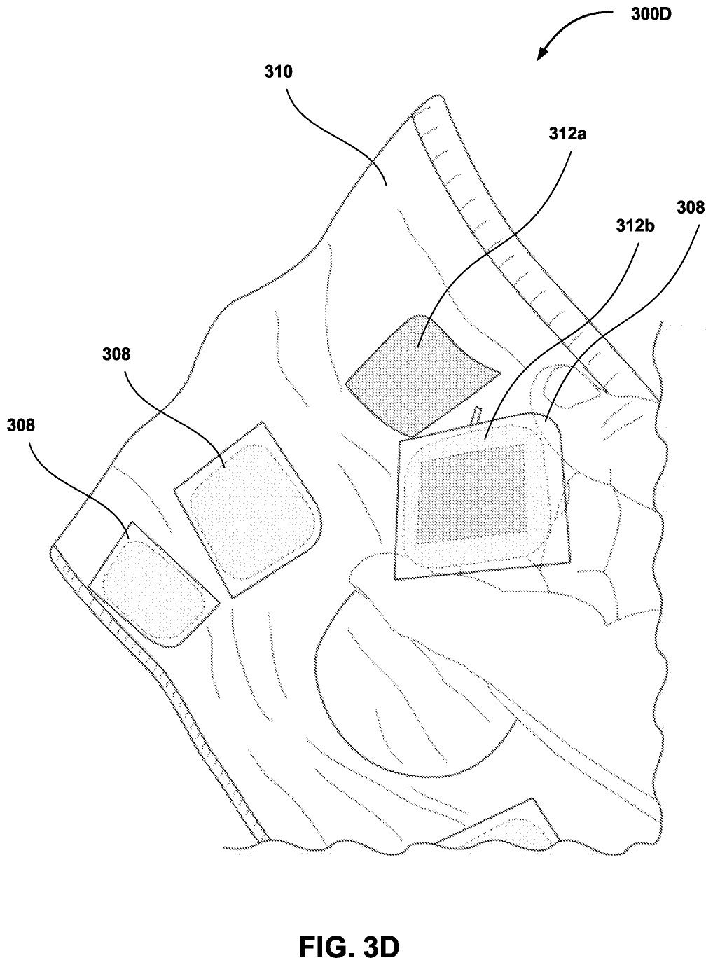

FIGS. 3A-3D are diagrams 300A-300D that illustrate an exemplary wearable device 100 developed to treat a painful or uncomfortable body part of a user, according to an exemplary embodiment of the present invention. In the given example, the body part has been considered the frozen shoulder of the user without limiting the scope of the present invention. FIG. 3A is a diagram 300A that shows a user 302 (here, a hand of the user has been shown) wearing the wearable device 100 around his hand. The wearable device 100 is attached or tied to the user's hand 302 using straps 304 of the wearable device 100. These straps 304 may allow it to be properly positioned and then tightened into place as shown. FIG. 3B is a diagram 300B that shows the wearable device 100 laid flat, with the electrodes facing a table surface. The exposed wires 306 are electrical leads that are connected to the electrodes. The wires are configured along all sides of the fabric and closure system that bridge electrical connections under the fabric. The wires may be inside material construction layers that is not visible to a naked eye. The electrodes may be disposable because the conductive adhesive tends to wear off over several uses. In case the existing electrodes have been disposed, new electrodes may be used with the disclosed wearable device 100. FIG. 3C is a diagram 300C that shows the strapping system 304 that allows the wearable device 100 to be tightened into place. FIG. 3D is a diagram 300D that shows the electrode 308 and the method for attaching it to the wearable device 100. In an embodiment, a hook and loop system 312a and 312b is used, wherein the hook portion 312b is adhered to the back of the electrode 308, and it can be mated to the loop portion 312a that is sewn into the sleeve 310. This allows the electrodes to be replaced with new electrodes as needed.

As shown in FIGS. 3A-3C, the wearable device 100 is a sleeve that has been developed to treat the frozen shoulder which is a painful chronic shoulder disorder. The device 100 is based on a “TENS unit” which uses electrode pairs to create muscle and nerve stimulation as the electrodes are applied to the surface of the user's skin, and a controller is used to send electric pulses of defined amplitude and duration through the various electrode pairs. This device 100 has been designed to be a sleeve that can be easily put on and it will have a large variety of pre-programmed stimulation patterns. The invention is designed to allow the controller to create various pairs between all of the electrodes in the array. One critical piece of the puzzle is that the TENS electrodes use a conductive adhesive to ensure a conductive connection with the skin. This device may require a “conductive media” be applied to each of the electrodes before use. In the disclosed device 100, any electrode may be configured to create a circuit with any of other electrodes of the same device 100. The primary purpose of this device 100 is to stimulate nerves and other structures in the epidermal and dermal layers, which will not go deeper into muscular structures. Muscular stimulation may be a by-product of this primary purpose and could be activated in concert with the primary purpose to achieve the stated goals of pain reduction. This device 100 may be primarily used for electrical nerve stimulation along with other structures in the epidermal and dermal layers. This may include stimulation that is just across or within the dermis. This device 100 has been designed that can be put on with minimal preparation, as well as being less cumbersome to wear due to the ability to harness wiring. Thus, the device is very easy to apply and use. It may require the application of a conductive media such as gel, but it is generally designed to allow a user to put on the device and to sleep comfortably while wearing it.

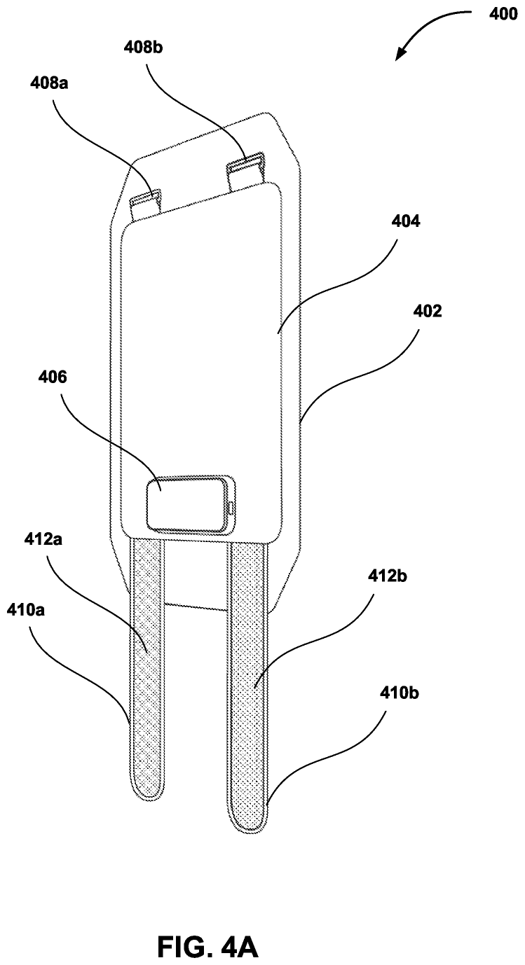

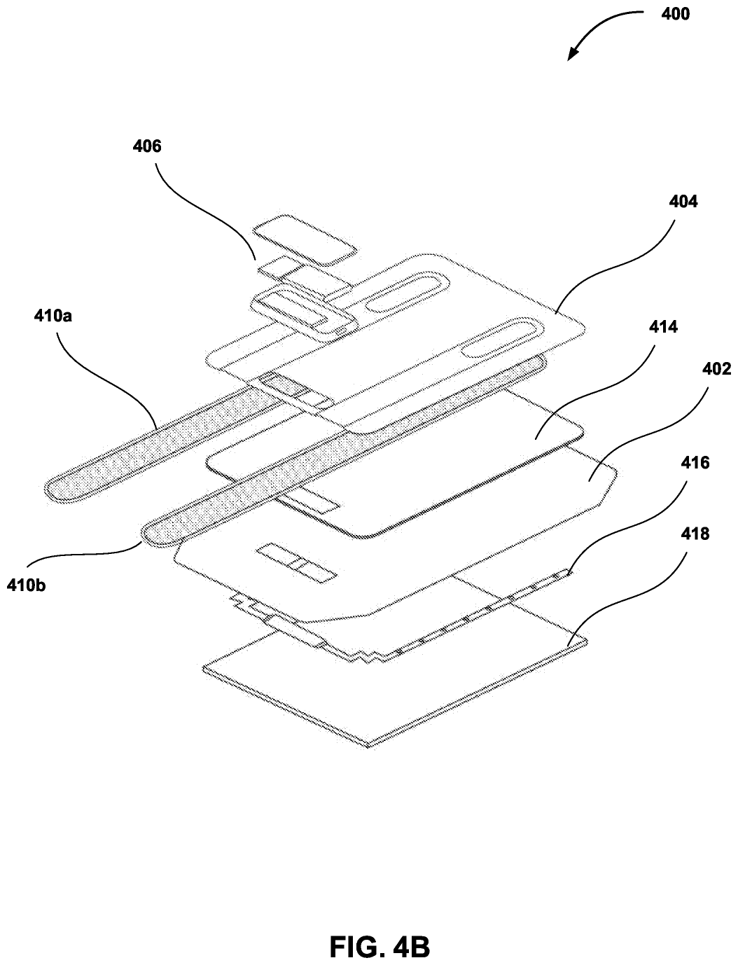

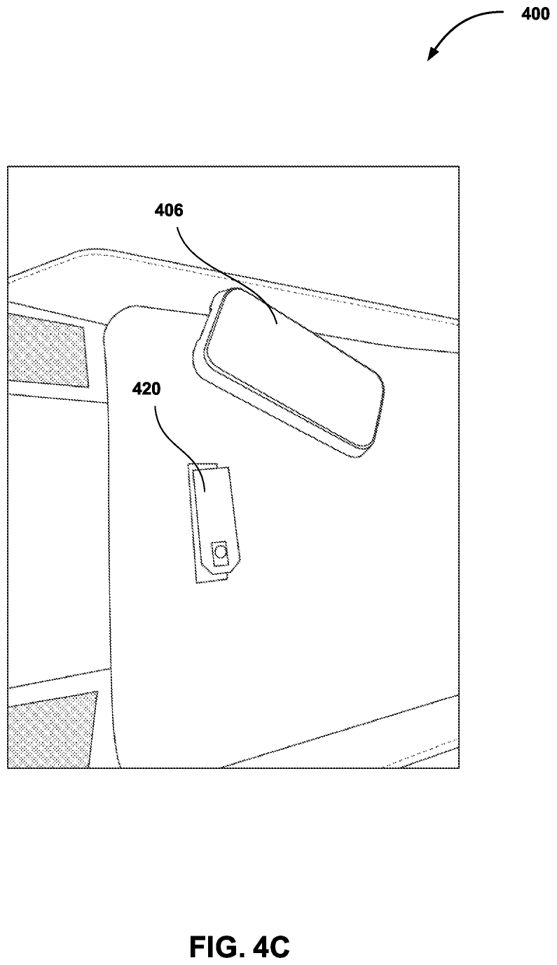

FIG. 4A is a diagram that illustrates a wearable device 400 to ameliorate pain, according to another exemplary embodiment of the present invention. The wearable device 400 includes a fabric, a closure system, a battery set, a charging port, a processor, a transmitter/receiver system, movement sensors, and an electrical system. The primary purpose of this device 100 is to stimulate nerves and other structures in the epidermal and dermal layers, which will not go deeper into muscular structures. The wearable device 400 includes a foldable band strip or fabric 402 that is made up of neoprene or soft touch material that can be folded or wrapped around a body portion such as wrist, elbow, shoulder, ankle, calf, knee, thigh, lower back, neck, or the like. The foldable band strip 402 may be shaped to fit around an extremity of a user or a body section. The foldable band strip 402 is made of a flexible elastomeric material which, when applied, draws snugly and conforms about the contours of such extremity or body section. The foldable band strip 402 may have the ability to draw snugly around most extremities or locations on account of its closure system. The closure system is a fastener, for example, a version of the hook-and-loop fastener, more commonly known as Velcro®. The fit of the foldable band strip 402, and hence the functionality of the wearable device 400 may be dependent on the closure system. There are other closure devices that may be implemented for varying locations and use case scenarios. The wearable device 400 further includes a top cover 404 for aesthetic purpose. A module assembly 406 is attached to a top surface of this cover 404. The module assembly 406 is a 3D printed connector sensor that is attached to the top surface of the cover 404. There is further provided a plurality of Velcro ends or hooks 408a and 408b and a plurality of bands or straps 410a and 410b with or without Velcro strips 412a and 412b. After wrapping the device 400 around a specific body part, the straps 410a and 410b are passed through or inserted into the hooks 408a and 408b to tighten the roll around of the device 400 around the body part.

FIG. 4B is a diagram that illustrates an exploded version of the wearable device 400, according to another exemplary embodiment of the present invention. The wearable device 400 includes the foldable band strip or fabric 402 and the aesthetic cover 404. The wearable device 400 further includes the module assembly 406 and the straps 410a and 410b. The wearable device 400 further includes a support material 414, a flexible electrical contact 416, and a hydrogel pad 418. The electrical contact 416 includes the conductive pathway system. The conductive pathway system may be located on outside and underside the fabric. The conductive pathway system may allow signals to be sent from the processor to one side of the device. Various portions and components (as shown in FIG. 4B) are assembled together to form the wearable device 400. The wearable device 400 may further include a battery set including one or more rechargeable batteries that are located on within the fabric to maximize comfort while in use. The battery may have the ability to be recharged over the lifespan of the wearable device 400. The battery may be connected to a charging port that will allow the battery to be recharged multiple times over the lifespan of the wearable device 400. Although not shown, a charging cable may be provided that connects to the battery when not in use and is sold along with the device 400. The charging cable may be used to power the battery from an external power supply, which in turn charges the battery when not in use. The battery may be configured to power the wearable device 400 in general including all of its communicating, sensing, processing, and stimulation functionalities such as one or more processors to handle input/output (I/O) from/to a mobile application or controller, one or more transmitters, one or more receivers, and one or more motion sensors. The wearable device 400 further includes a processor. The processor may be located on the outside of the fabric to maximize comfort while in use. The processor may be powered by the battery. The processor proves integral in the functionality of the wearable device 400. The ability of the processor may include, but is not limited to, sending signals, recognizing and interpreting movement data, compiling and managing movement data, and communicating with transmitter/receiver system. For example, the processor may be configured to receive directive(s) from a transmitter of the transmitter/receiver system. The processor may be further configured to interpret the directive(s) and organize stimulation accordingly. The processor may be further configured to send the stimulation to chosen wires. The processor may be further configured to terminate stimulation when the circuit is broken. The processor additionally monitors the health of the battery in order to properly notify the user when there is a need of charging or replacement, depending on the version of the battery. The processor may be further configured to gather and send a battery charge report and the set of sleeping patterns from internal recording or via third-party integration. The processor may be further configured to vary the energy, pulse width, frequency, duration, and current of the stimulation. The processor may be further configured to deliver a specific dermal stimulation mode through a wireless transmission from a signal originating from a mobile device application or remote via the transmitter/receiver system. The processor may be further configured to implement a mode selected from a plurality of modes consisting of a basic on and off setting, a mode consisting of a timed and timer setting, a mode consisting of using the movement sensing system, a mode communicating with a third-party wearable device, and others. The processor may be further configured to communicate with the third-party wearable device. The wearable device contains the ability to track movement of the user. In an embodiment, the processor contains the ability to provide feedback personalized to the user about health, sleep, and movement presented through a mobile device application or remote. The wearable device 400 further includes multiple movement sensors. The movement sensors may be located on the outside of the fabric to maximize comfort while in use and is powered by the battery. The amount of movement sensors depend on the various embodiments the device takes. Regardless of the specific amount of movement sensors, the purpose of these sensors is to detect the movement of the user while the wearable device 400 is both attached to the user and functioning in a certain mode, both conditions relate to the exemplary embodiment of the wearable device 400. Although the main purpose of the sensors and accompanying mode carried out via smart product application or remote is to monitor sleep cycle patterns and correspond said cycle with proper stimulation in order to limit pain experienced by the user, it can generally be used for occasions when the user anticipates a period of inactivity for corresponding pain area. In general, the multiple movement sensors detect user body movement data. Such detected data is sent to the processor which in turn translates the data into decisions based on the hardware, processor, and software of the mobile device application or remote. Such data is both simultaneously translated into a change of power and/or frequency and/or pattern of stimulation. The data is sent to the transmitter, relaying the change of power to the smart product application or remote. The movement sensor system has been created in order to reduce the effects that the users may experience when they are immobile for long periods of time. The system is designed to aid the user for sleeping or relaxation, in this exemplary embodiment. The stimulation would allow the user to enter or re-enter a state of relaxation and/or sleep. Once the movement sensor system would detect a lack of user movement, the stimulation would gradually decrease power and/or change frequency and/or pattern. A quiet period would ensue when the stimulation would cease caused by the processor, however the movement sensor system would remain active. Once the movement sensor system would detect a change in the immobility, usually characterized by the later stages of the sleep cycle, the system would utilize the processor to phase the stimulation in, gradually increasing power. This could also be linked to third-party applications that log, categorize, and predict wakefulness and sleep patterns. This would, in essence prime the affected area for movement after a prolonged immobile interval. In the exemplary embodiment of the device 400, the multiple movement sensors are instrumental in the movement sensing system. In some embodiments, the signals may be randomly distributed along the body part by changing the connection of various electrodes. This mechanism may be referred to as the random nerve stimulation that may be critical and useful to pain alleviation. The random nerve simulation may be achieved by preforming the rotation of various electrode pairs and firing them based on their connection.

FIG. 4C is a diagram that illustrates a use case scenario of using the wearable device 400 to ameliorate pain, according to another exemplary embodiment of the present invention. In this case, the module assembly 406 has been removed from its designated location. Before using the device, the assembly 406 is attached to a connector 420.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

What is claimed is:

1. A wearable device for simulating physical sensation, the wearable device comprising:
a fabric and closure system for securing to a user;
a configuration featuring wires inside and around all sides of the fabric and closure system that bridge electrical connections, wherein the wires are not visible to a naked eye;
a battery that powers the device;
a processor configured to:
receive directive(s) from a transmitter,
interpret the directive(s) and organize stimulation accordingly,
send the stimulation to chosen wires,
terminate the stimulation when the circuit is broken, and
gather and send a battery charge report associated with the battery and a set of sleeping patterns from internal recording or via third-party integration; and
a transmitter and receiver for remotely communicating with a mobile device application relaying a user selection of a plurality of operating modes,
wherein the operating directive corresponds to the selected mode and includes at least one of a plurality of pulse frequencies, a plurality of pulse-widths, a plurality of durations, or a plurality of signals.

2. The wearable device of claim 1, wherein the fabric is made of a flexible elastomeric material which, when applied, draws snugly and conforms about contours of such extremity or body section.

3. The wearable device of claim 1, wherein the closure system comprises a hook-and-loop fastener, a dual lock fastener, or a tension strap system.

4. The wearable device of claim 1, wherein the battery is a rechargeable battery that is located outside of the fabric.

5. The wearable device of claim 4, wherein the battery is configured to power the device in including all of its communicating, sensing, processing, and stimulation functionalities associated with one or more processors to handle input/output (I/O) from/to a mobile application or controller, one or more transmitters, one or more receivers, and one or more motion sensors.

6. The wearable device of claim 1, wherein the processor is located outside of the fabric to maximize comfort while in use.

7. The wearable device of claim 6, wherein the processor is further configured to monitor the health of the battery in order to properly notify a user when there is a need for charging or replacement of the battery.

8. The wearable device of claim 7, wherein the processor is further configured to vary energy, pulse width, frequency, duration, and current of the stimulation.

9. The wearable device of claim 8, wherein the processor is further configured to implement a mode selected from the plurality of modes consisting of a basic on and off setting, a mode consisting of a timed and timer setting, a mode consisting of using the movement sensing system, and a mode communicating with a third-party wearable device.

10. The wearable device of claim 1, further comprising movement sensors that are located outside of the fabric to maximize comfort while in use and is powered by the battery.

11. The wearable device of claim 1, wherein the movement sensors are configured to detect user body movement data of a user while the device is both attached to the user and functioning in a certain mode.

12. A wiring system for a wearable device simulating physical sensation, the wiring system comprising:
an arrangement of wires inside and around all sides of a fabric;
a battery that supplies energy; and
a processor configured to send stimulation across chosen wires, wherein the processor is further configured to receive directive(s), respond to said directive(s) by delivering corresponding stimulation, and is powered by the battery.
